(19) United States
(12) Patent Application Publication
(10) Pub. No.: US 2023/0226011 A1
(43) Pub. Date: Jul. 20, 2023

(54) Novel Pharmaceutical Composition Comprising Combination of Olopatadine and Nepafenac

(71) Applicant: Zenvision Pharma LLP, Mumbai (IN)

(72) Inventors: Sivakumar Venkata Bobba, Navi Mumbai (IN); Amit Manmode, Navi Mumbai (IN); Dhananjay Shinde, Navi Mumbai (IN); Sunil Pophale, Navi Mumbai (IN); Shankar Pol, Navi Mumbai (IN)

(21) Appl. No.: 18/007,866

(22) PCT Filed: Jun. 4, 2021
(86) PCT No.: PCT/IB2021/054900

§ 371 Date: Dec. 2, 2022

(30) Foreign Application Priority Data
Jun. 4, 2020 (IN) 201921049889

(51) Int. Cl. A61K 31/335; A61K 31/165; A61K 9/00; A61K 47/10; A61K 47/26; A61K 47/02; A61K 9/06

(52) U.S. Cl. CPC A61K 31/335; A61K 31/165; A61K 9/0048; A61K 47/10; A61K 47/26; A61K 47/02; A61K 9/06

(57) ABSTRACT

The present invention relates to a novel pharmaceutical composition comprising combination of Olopatadine or salt thereof and Nepafenac or salt thereof, process for preparation thereof and use thereof for the treatment of ophthalmic disorders.

### FIELD OF THE INVENTION

The present invention relates to a novel pharmaceutical composition comprising combination of Olopatadine and Nepafenac for the treatment of ophthalmic disorder.

The present invention further relates to a process for preparation novel pharmaceutical composition comprising combination of Olopatadine or salt thereof and Nepafenac or salt thereof.

The present invention particularly relates to an ophthalmic pharmaceutical composition comprising combination of Olopatadine or salt thereof and Nepafenac or salt thereof, process of preparation thereof and use of the said composition in the treatment of ophthalmic disorder.

### BACKGROUND OF THE INVENTION

Olopatadine is a mast cell stabilizer and a histamine H1 antagonist. Chemically Olopatadine hydrochloride is 11-[(Z)-3(Dimethylamino) propylidene]-6-11-dihydrodibenz[b,e] oxepin-2-acetic acid, hydrochloride and its molecular weight is of 373.88. Its empirical formula is C₂₁H₂₃NO₃·HCl. Olopatadine hydrochloride is represented by compound of structural formula I

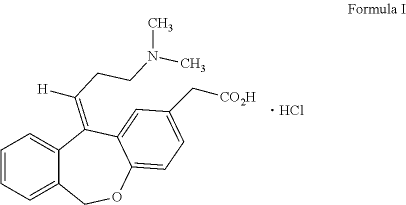

Olopatadine hydrochloride is white crystalline powder, sparingly soluble in methanol and water and insoluble in chloroform.

Olopatadine hydrochloride ophthalmic solution has been approved in USA as on 5 Dec. 18, 1996 under the trade name PATANOL® and is available in the strength of 0.1% base. Further Olopatadine hydrochloride ophthalmic solution has been approved in USA as on Dec. 22, 2004 and Jan. 30, 2015 under the trade name PATADAY® and PAZEO® and is available in the strength of 0.2% base and 0.7% base respectively.

NSAID's such as Nepafenac is a non-steroidal anti-inflammatory prodrug. Chemically Nepafenac is 2-amino-3-benzoylbenzeneacetamide and its molecular weight is of 254.28. Its empirical formula is C₁₅H₁₄N₂O₂. Nepafenac is represented by compound of structural formula II

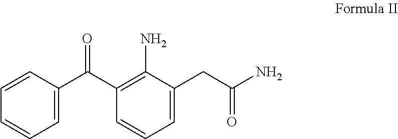 [SMILES: NC(=O)Cc1cccc(C(=O)c2ccccc2)c1N]

Nepafenac is a yellow crystalline powder and practically insoluble in water

Nepafenac ophthalmic suspension has been approved in USA as on Aug. 19, 2005 under the trade name NEVANAC® and is available in the strength of 0.1%. Also the Nepafenac ophthalmic suspension has been approved in USA as on Oct. 16, 2012 under the trade name ILEVRO® and is available in the strength of 0.3%.

Nepafenac ophthalmic suspension i.e. NEVANAC® and ILEVRO® both has been used for the treatment of pain and inflammation associated with cataract surgery.

Olopatadine hydrochloride ophthalmic solution i.e. PATANOL® is used for the treatment of the signs and symptoms of allergic conjunctivitis. Olopatadine hydrochloride ophthalmic solution i.e. PATADAY® and PAZEO® are used for the treatment of treatment of ocular itching associated with allergic conjunctivitis

U.S. Patent Publication No. 20130281506 discloses generically composition comprising a combination of a non-steroidal anti-inflammatory drug with an antihistamine drug intended for an ophthalmic use. This patent publication particularly related to combination of ketorolac (non-steroidal anti-inflammatory drug) with olopatadine (anti-histaminic drug) for treatment of seasonal ocular surface allergy.

Florida Administrative code and Florida administrative register, section 64B13-18.002 Formulary of Topical Ocular Pharmaceutical Agents discloses generically “alone or combination of cycloplegic and mydriatics, local anesthetics, diagnostic products, antibacterial, NSAID, antihistamines, antiviral, antifungal and anti-glaucoma agents for the ophthalmic disorder”.

Eyes are the critical sensory organ of the human being and are associated with several ophthalmic disorders. Many ophthalmic disorders do not cure or provide patient compliance by use of antihistamine/mast cell stabilizer alone or non-steroidal anti-inflammatory drug alone. Such ophthalmic disorders are associated with allergy, inflammation, pain, release of histamines from the cell. Therefore, to treat these ophthalmic conditions requires combination of antihistamine/mast cell stabilizers along with non-steroidal anti-inflammatory drug.

The commercially available products and products known in the prior art does not treat these ophthalmic disorder effectively and does not provide patient compliance in the treatment of the same.

So, there is need in the art to provide such combination of antihistamine/mast cell stabilizer along with non-steroidal anti-inflammatory drug in the treatment of ophthalmic disorders associated with allergy, inflammation, pain, release of histamines from the cell.

Accordingly, the applicant of the present invention invented novel pharmaceutical composition comprising combination of Olopatadine and Nepafenac, which are more efficacious and provides better patient compliance in the treatment of these ophthalmic disorder.

### OBJECT OF THE INVENTION

Accordingly, it is another object of the present invention to provide a novel pharmaceutical composition comprising:

a) Olopatadine or salt thereof;
b) Nepafenac or salt thereof; and
c) at least one suitable pharmaceutically acceptable excipient.

It is another object of the present invention to provide a novel ophthalmic pharmaceutical composition comprising:

a) Olopatadine or salt thereof; and
b) Nepafenac or salt thereof; and
c) at least one pharmaceutically acceptable excipient.

It is another object of an invention provides process for the preparation of a novel pharmaceutical composition, wherein process comprises steps of:

a) Dissolving and/dispersing excipients in part of solvent;
b) Dissolving Olopatadine or salt thereof in part of solvent and mixing it to solution/dispersion of step (a);
c) Dispersing Nepafenac or salt thereof in part of solvent and mixing it to solution/dispersion of step (b); and
e) Adjusting the volume with remaining quantity of solvent.

It is another object of the present invention to provide a novel and commercially viable process of preparation of a novel ophthalmic pharmaceutical composition comprising combination of Olopatadine or salt thereof and Nepafenac or salt thereof.

It is another object of the present invention to provide method of treating ophthalmic diseases by administrating a novel ophthalmic pharmaceutical composition comprising combination of Olopatadine or salt thereof and Nepafenac or salt thereof.

### SUMMARY OF THE INVENTION

A first aspect of the present invention is to provide a novel pharmaceutical combination of Olopatadine and Nepafenac.

In another aspect of the present invention is to provide a novel pharmaceutical composition comprising combination of Olopatadine and Nepafenac.

In another aspect of the present invention is to provide a novel pharmaceutical ophthalmic composition comprising combination of Olopatadine and Nepafenac.

In another aspect of the present invention is to provide a novel pharmaceutical ophthalmic composition comprising combination of Olopatadine and Nepafenac along with one or more pharmaceutically acceptable excipient.

In another aspect, an invention provides a novel pharmaceutical ophthalmic composition comprising combination of Olopatadine or salt thereof and Nepafenac or salt thereof along with and at least one pharmaceutically acceptable excipient including penetration enhancer or surfactants, antimicrobial agent, preservative, antioxidant, tonicity agent, vehicle, gelling agent, thickening agent, cosurfactant, humectant, acidifying or alkalizing or buffering agent, absorbent, chelating agent, opacifying agent or combination thereof in a sufficient concentration to provide a stable composition.

In another aspect, an invention provides a novel pharmaceutical composition comprising:

a) Olopatadine or salt thereof;
b) Nepafenac or salt thereof; and
c) at least one suitable pharmaceutically acceptable excipient.

It is another aspect, an invention provides a novel ophthalmic pharmaceutical composition comprising:

a) Olopatadine or salt thereof;
b) Nepafenac or salt thereof; and
c) at least one pharmaceutically acceptable excipient.

In another aspect of the present invention is to provide process of preparing a novel pharmaceutical ophthalmic composition comprising combination of Olopatadine and Nepafenac along with one or more pharmaceutically acceptable excipient.

In another aspect of the present invention is to provide process for the preparation of a novel pharmaceutical composition, wherein process comprises steps of:

a) Dissolving and/dispersing excipients in part of solvent;
b) Dissolving Olopatadine or salt thereof in part of solvent and mixing it to solution/dispersion of step (a);
c) Dispersing Nepafenac or salt thereof in part of solvent and mixing it to solution/dispersion of step (b);
e) Adjusting the volume with remaining quantity of solvent.

In another aspect of the present invention is to provide a novel pharmaceutical composition comprising combination of Olopatadine and Nepafenac in the treatment of ophthalmic disorders.

In another aspect of the present invention is to provide a novel pharmaceutical ophthalmic composition comprising combination of Olopatadine or salt thereof and Nepafenac or salt thereof in the treatment of ophthalmic disorders.

In another aspect of the present invention is to provide a novel pharmaceutical composition comprising combination of Olopatadine or salts as antihistamines thereof and Nepafenac or salts thereof as non-steroidal anti-inflammatory drug in the treatment of adverse clinical symptom associated with use of contact lens.

### DETAIL DESCRIPTION OF THE INVENTION

The term “composition”, “novel pharmaceutical composition” or “pharmaceutical composition” or “ophthalmic pharmaceutical composition” used herein interchangeably and is intended to encompass a drug product comprising Olopatadine or its pharmaceutically acceptable salts thereof and Nepafenac or its pharmaceutically acceptable salts thereof and other inert ingredient(s) (pharmaceutically acceptable excipients).

The term “Olopatadine” is used in broad sense to include not only “Olopatadine” per se but also its pharmaceutically acceptable salts, solvates, hydrates, nantiomers, derivatives, isomers, polymorphs, prodrugs thereof.

The term “Nepafenac” is used in broad sense to include not only “Nepafenac” per se but also its pharmaceutically acceptable base, salts, solvates, hydrates, nantiomers, derivatives, isomers, polymorphs, prodrugs thereof.

The term “pharmaceutically acceptable salt” refers to salts derived from a variety of organic and inorganic counter ions including hydrochloride, fumarate, maleate, phosphate, L-tartrate, citrate, acetate, oxalate, and sulfate or the like.

The term “excipient”, means a pharmacologically inactive component such as penetration enhancer or surfactants, preservative, antioxidant, vehicle, gelling agent, thickening agent, cosurfactant, humectant, acidifying or alkalizing or buffering agent, absorbent, chelating agent, opacifying agent or the like. The excipients that are useful in preparing a pharmaceutical composition are generally safe, non-toxic and are acceptable for veterinary as well as human pharmaceutical use. Reference to an excipient includes both one and more than one such excipient.

The term “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder or delaying, inhibiting or decreasing the likelihood of the progression of the disease, disorder or syndrome.

The term “stable” as used herein refers to formulations that substantially retain the label amount of the therapeutically active ingredient during storage for commercially relevant times, and the drug-related impurity contents in the formulations remain within the acceptable limit.

Throughout this specification and the appended claims, it is to be understood that the words “comprise”, “have”, “contain” and “include” and variations such as “comprises”, “comprising”, “having”, “containing” “includes”, “including” are to be interpreted inclusively, unless the context requires otherwise. That is, the use of these words may imply the inclusion of an element or elements not specifically recited.

The present invention relates to novel pharmaceutical combination of Olopatadine and Nepafenac in the treatment of ophthalmic disorders.

The active ingredient Olopatadine in the present invention may be in the form of Olopatadine base or its pharmaceutically acceptable salt. Olopatadine salt may be selected from but not limited to hydrochloride, acetate, maleate, fumarate, tartrate, citrate, magnesium, calcium salt.

The present invention preferably contains Olopatadine hydrochloride salt.

The active ingredient Nepafenac in the present invention may be in the form of Nepafenac base or its pharmaceutically acceptable salt. The present invention preferably contains Nepafenac base.

The ophthalmic disorders in the present invention may be selected from but not limited to conjunctivitis, ocular itching associated with allergic conjunctivitis, pain and inflammation associated with cataract surgery, adverse clinical symptom associated with use of contact lens, Blepharitis, Blepharoconjunctivitis, Cytomegalovirus retinitis, Corneal ulcer, Herpes Simplex Dendritic Keratitis, Herpetic Keratitis, Hordeolum, Keratitis, Keratoconjunctivitis, Neonatal Conjunctivitis, Ophthalmic Surgery, Trachoma, Nearsightedness (myopia), Farsightedness, Astigmatism, Presbyopia, Foreign object in the eye, Cataract, Eye floaters, Corneal abrasion, Eye redness, Dry eyes, Visual disturbances, External eyelid stye, Watery eyes, Eye pain, entropion, Eye burning accompanied by itching and discharge, Eyelid twitch, Glaucoma, Dry eye syndrome, Black eye, subconjunctival hemmorhage, Retinal detachment, Lazy eye, Pterygium, Diabetic retinopathy, Night blindness, Eye emergencies, Visual impairment, Hypertensive retinopathy, Strabismus, Age related macular degeneration, Bulging eyes, Uveitis, Scleritis, Photophobia, Hyphema, Ophthalmoplegia, Ocular migraines, Sarcoidosis, Retinal vascular occlusion, Optic neuritis and the like.

The ophthalmic disorders in the present invention preferably may be selected from but not limited to signs and symptoms of allergic conjunctivitis, ocular itching associated with allergic conjunctivitis, pain and inflammation associated with cataract surgery, adverse clinical symptom associated with use of contact lens.

The olopatadine is an inhibitor of the release of histamine from the mast cell and a relatively selective histamine H1-antagonist that inhibits the type 1 immediate hypersensitivity reaction including inhibition of histamine induced effects on human conjunctival epithelial cells.

The non-steroidal anti-inflammatory drug Nepafenac for treating ophthalmic disorders provides their action by inhibiting the activity of Cyclooxygenase, an enzyme required for prostaglandin synthesis.

The novel pharmaceutical combination of olopatadine which act by blocking histamine H1 at the receptor site, inhibiting histamine release from the mast cell and Nepafenac which act by inhibiting the activity of Cyclooxygenase effectively treat ophthalmic disorders.

The concentration in the novel pharmaceutical combination of Olopatadine or its salt and Nepafenac or its salt provides effective and/synergistic treatment in the ophthalmic disorders with better patient compliance. The compositions contain 0.05 to 2% (w/v) Olopatadine or its salt, more specifically composition contains 0.1 to 1% (w/v) Olopatadine or its salt. The compositions contain 0.01-1.5% (w/v) Nepafenac or its salt, more specifically composition contains 0.05 to 1% (w/v) Nepafenac or its salt.

In another aspect of the present invention is to provide novel pharmaceutical composition comprising combination of Olopatadine and Nepafenac in the treatment of ophthalmic disorders.

The novel pharmaceutical combination of Olopatadine and Nepafenac preferably may be in the form of ophthalmic composition.

The novel pharmaceutical ophthalmic composition comprising combination of Olopatadine and Nepafenac for the treatment of ophthalmic disorders may be in the form of Eye drop, Solution, Suspension, Microemulsions, Ointments, Lotions, Gels, Injections, Nanoparticles, Liposomes, Niosomes, Hydrogels and the like.

The novel pharmaceutical ophthalmic composition comprising combination of Olopatadine and Nepafenac along with pharmaceutically acceptable excipient.

The novel pharmaceutical ophthalmic composition comprising combination of Olopatadine and Nepafenac of the present invention may contain one or more excipient selected from the group consisting of gelling agent, solubilizers, buffering agents, tonicity agents, viscosity enhancers, antimicrobial agent, preservatives, antioxidizing agents, pH regulators, chelating agents, surfactants and vehicles.

The examples of solubilizers include but not limited to polyethylene glycol, polyoxyethylene monostearate, polyoxyethylene hydrogenated castor oil, polysorbate 80, cyclodextrin or the like or combination thereof.

The examples of buffering agents include but not limited to carbonate buffers, borate buffers, phosphate buffers, acetate buffers, citrates, gluconates, lactates, propionates and tromethamine buffers or the like or combination thereof.

The examples of tonicity agents include but not limited to mannitol, sorbitol, glycerin, sodium chloride and other electrolytes or the like or combination thereof.

The examples of viscosity enhancers include but not limited to carboxymethylcellulose, methylcellulose, hydroxypropylmethylcellulose, hydroxyethylcellulose, polyvinyl alcohol, chondroitin sulfate or the like or combination thereof.

The examples of antimicrobial preservatives include but not limited to benzalkonium chloride, benzethonium chloride, parahydroxybenzoic acid esters, cetyl pyridinium chloride, sodium dehydroacetate, phenethyl alcohol, chlorobutanol, phenylmercuric acetate, phenylmercuric nitrate or the like or combination thereof.

The examples of antioxidizing agents include but not limited to sodium bisulfite, sodium metabisulfite, ethylenediaminetetraacetic acid, thiourea or the like or combination thereof.

The examples of pH regulators include but not limited to citric acid, acetic acid, hydrochloric acid, phosphoric acid, sodium carbonate, sodium hydroxide, potassium hydroxide or the like or combination thereof.

The examples of chelating agents include but not limited to EDTA and its salts, such as but not limited to Disodium Edetate or the like or combination thereof.

The examples of surfactants include but not limited to tyloxapol, poloxamers, sorbitan esters, polyoxyl stearates, polysorbates, polyoxyethylene castor oil derivatives or the like or combination thereof.

The examples of vehicle include but not limited to sterile purified water, oils such as olive oil, castor oil, sesame oil or the like or combination thereof.

The examples of gelling agent include but not limited to Carbomer 974P, Carbomer, sodium alginate, cellulose derivative(s) or the like or combination thereof.

In another aspect of the present invention is to provide process of preparing novel pharmaceutical ophthalmic composition comprising combination of Olopatadine and Nepafenac along with pharmaceutically acceptable excipient.

The novel pharmaceutical ophthalmic composition of the present invention may be prepared by the process known in the art for ophthalmic products.

In another aspect of the present invention is to provide novel pharmaceutical composition comprising combination of Olopatadine and Nepafenac in the treatment of ophthalmic disorders.

The novel pharmaceutical composition comprising combination of Olopatadine and Nepafenac is used in the treatment of ophthalmic disorders selected from Conjunctivitis, ocular itching associated with allergic conjunctivitis, pain and inflammation associated with cataract surgery, adverse clinical symptom associated with use of contact lens, Blepharitis, Blepharoconjunctivitis, Cytomegalovirus retinitis, Corneal Ulcer, Herpes Simplex Dendritic Keratitis, Herpetic Keratitis, Hordeolum, Keratitis, Keratoconjunctivitis, Neonatal Conjunctivitis, Ophthalmic Surgery, Trachoma, Nearsightedness (myopia), Farsightedness, Astigmatism, Presbyopia, Foreign object in the eye, Cataract, Eye floaters, Corneal abrasion, Eye redness, Dry eyes, Visual disturbances, External eyelid stye, Watery eyes, Eye pain, entropion, Eye burning accompanied by itching and discharge, Eyelid twitch, Glaucoma, Dry eye syndrome, Black eye, subconjunctival hemmorhage, Retinal detachment, Lazy eye, Pterygium, Diabetic retinopathy, Night blindness, Eye emergencies, Visual impairment, Hypertensive retinopathy, Strabismus, Age related macular degeneration, Bulging eyes, Uveitis, Scleritis, Photophobia, Hyphema, Ophthalmoplegia, Ocular migraines, Sarcoidosis, Retinal vascular occlusion and Optic neuritis

The novel pharmaceutical composition comprising combination of Olopatadine and Nepafenac is preferably used in the treatment of signs and symptoms of allergic conjunctivitis, ocular itching associated with allergic conjunctivitis, pain, inflammation associated with cataract surgery and adverse clinical symptom associated with use of contact lens.

In another aspect of the present invention is to provide novel pharmaceutical composition comprising combination of Olopatadine or salt thereof as antihistamines and Nepafenac or salt thereof as non-steroidal anti-inflammatory drug in the treatment of adverse clinical symptom associated with use of contact lens.

The novel pharmaceutical ophthalmic composition of the present invention may be filled in to the single dose container or multiple dose containers and packaged into the plastic or glass containers.

In another embodiment, ophthalmic pharmaceutical composition of Olopatadine or salt thereof and Nepafenac or salt thereof according to present invention can be packaged into the suitable container like collapsible tubes, jars, pot, bottle or single dose packets or any suitable packaging material. The container material or packaging material of the present invention does not affect the quality of the preparation or does not allow diffusion of any kind into or across the material of the container into the preparation.

In another embodiment, an invention provides the process for the preparation of a novel pharmaceutical composition, wherein process comprises steps of:

a) Dissolving and/dispersing excipients in part of solvent;
b) Dissolving Olopatadine or salt thereof in part of solvent and mixing it to solution/dispersion of step (a);
c) Dispersing Nepafenac or salt thereof in part of solvent and mixing it to solution/dispersion of step (b); and
e) Adjusting the volume with remaining quantity of solvent.

In another embodiment, an invention provides the process for the preparation of a novel ophthalmic pharmaceutical composition, wherein process comprises steps of:

a) Dissolving/dispersing excipients like Mannitol, Sodium chloride, and Disodium Edetate in part of solvent;
b) Dispersing gelling agent excipients to form homogenous dispersion of Gel polymer;
c) Step (a) solution was mixed with step (b), Gel polymer dispersion;
d) Dissolving Olopatadine or salt thereof in part of solvent mixing it to solution;
e) Transfer Olopatadine or its salts solution of step (d) into dispersion of step (c);
f) Dispersing Nepafenac or salt thereof in part of solvent and homogenization continued for 30 minutes; and
g) Step (f) dispersion was transferred to step (e) and note down pH of the dispersion, further adjusting the volume with remaining quantity of solvent.

### EXAMPLE

The following example is provided solely for illustrative purposes and is not meant to limit the scope of the invention in any way.

### Example 1: With Gel Approach

| | | Composition | Composition |
|---|---|---|---|
| Sr. No. | Ingredients | % (w/v) | % (w/w) |
| 1. | Olopatadine Hydrochloride | 0.1 | 0.2 |
| 2. | Nepafenac | 0.05 | 0.1 |
| 3. | Carbomer 974P | 0.48 | 0.48 |
| 4. | Mannitol | 2.4 | 2.4 |
| 5. | Sodium chloride | 0.4 | 0.4 |
| 6. | Tyloxapol | 0.01 | 0.01 |
| 7. | Disodium Edetate | 0.01 | 0.01 |
| 8. | Benzalkonium chloride | 0.01 | 0.01 |
| 9. | Sodium hydroxide to | q.s. | q.s |
| | adjust pH to about 7.2 | | |
| 10. | Purified water | q.s. to 100 g | q.s. to 100 g |

### Manufacturing PROCESS:

1. Mannitol, Sodium chloride, and Disodium Edetate were dissolved in part quantity of water (10%).
2. Carbomer 974P was dispersed in part quantity of water (60%) to form homogenous dispersion of Gel polymer.
3. Step (1) solution was mixed with step (2), Gel polymer dispersion.
4. The pH of step (3) dispersion was noted.
5. Dispersion of step (3) was autoclaved and cooled at room temperature, pH was noted.
6. Nitrogen flushing was continued in order remove oxygen from dispersion.
7. Olopatadine Hydrochloride was dissolved in part quantity of water, (20%) flushed with Nitrogen and temperature was maintained at 70-80° C.
8. Cooling: Solution of step (7) was cooled to room temperature and pH was adjusted to about 7.2
9. Filtration: Solution of step (8) was filtered through 0.22 micron filtered and pH was noted.
10. Transfer the solution of Step (9) to the dispersion of Step (3).
11. Tyloxapol was dispersed and dissolved in remaining part quantity of water (5%).
12. Nepafenac was dispersed in the above solution of Step (11) and homogenization continued for 30 minutes.
13. Step (11) dispersion was transferred to step (3). pH of the batch was noted.
14. Volume make was done upto 100 ml and further mixed for 30 minutes. pH of batch was noted.

1: A novel pharmaceutical composition comprising: a) Olopatadine or salt thereof; b) Nepafenac or salt thereof; and c) at least one suitable pharmaceutically acceptable excipient.

2: A novel pharmaceutical composition according to claim 1, wherein Olopatadine or salt thereof is preferably Olopatadine hydrochloride salt form.

3: A novel pharmaceutical composition according to claim 1, wherein Nepafenac is preferably used in Nepafenac base form.

4: A novel pharmaceutical composition according to claim 1, wherein composition is in the form of ophthalmic pharmaceutical composition.

5: A novel pharmaceutical composition according to claim 1, wherein a tonicity agents is selected from the group consisting of mannitol, sorbitol, glycerin, sodium chloride and other electrolytes or combination thereof.

6: A novel pharmaceutical composition according to claim 1, wherein a surfactants is selected from the group consisting of tyloxapol, poloxamers, sorbitan esters, polyoxyl stearates, polysorbates, polyoxyethylene castor oil derivatives or combination thereof.

7: A novel pharmaceutical composition according to claim 1, wherein chelating agents include but not limited to EDTA and its salts, preferably Disodium Edetate.

8: A novel pharmaceutical composition according to claim 1, wherein antimicrobial preservatives is selected from the group consisting of benzalkonium chloride, benzethonium chloride, parahydroxybenzoic acid esters, cetyl pyridinium chloride, sodium dehydroacetate, phenethyl alcohol, chlorobutanol, phenylmercuric acetate, phenylmercuric nitrate or combination thereof.

9: A novel pharmaceutical composition according to claim 1, wherein gelling agent include but not limited to Carbomer 974p, Carbomer derivatives, Sodium alginate, xanthan gum, cellulose derivatives and alike or combination thereof.

10: A novel pharmaceutical composition according to claim 1, wherein pH regulators is selected from the group consisting of citric acid, acetic acid, hydrochloric acid, phosphoric acid, sodium carbonate, sodium hydroxide, potassium hydroxide or combination thereof.

11: A novel pharmaceutical composition according to claim 1, wherein vehicle is selected from the group consisting of sterile purified water, oils such as olive oil, castor oil, sesame oil or combination thereof.

12: A process of preparation of a novel pharmaceutical composition, wherein process comprises steps of: a) Dissolving/dispersing excipients in part of solvent(s); b) Dispersing gelling agent excipients to form homogenous dispersion of Gel polymer; c) Step (a) solution was mixed with step (b), Gel polymer dispersion; d) Dissolving Olopatadine or salt thereof in part of solvent mixing it to solution; e) Transfer Olopatadine or its salts solution of step (d) into dispersion of step (c); f) Dispersing Nepafenac or salt thereof in part of solvent and homogenization continued for 30 minutes; and g) Step (f) dispersion was transferred to step (e) and note down pH of the dispersion, further adjusting the volume with remaining quantity of solvent.

13: A novel pharmaceutical composition according to claim 1, wherein the pharmaceutical composition is in the form of novel ophthalmic composition, preferably in the form of gel.

14: A novel pharmaceutical composition according to claim 2, wherein the pharmaceutical composition is in the form of novel ophthalmic composition, preferably in the form of gel.

15: A novel pharmaceutical composition according to claim 3, wherein the pharmaceutical composition is in the form of novel ophthalmic composition, preferably in the form of gel.

16: A novel pharmaceutical composition according to claim 4, wherein the pharmaceutical composition is in the form of novel ophthalmic composition, preferably in the form of gel.

17: A novel pharmaceutical composition according to claim 5, wherein the pharmaceutical composition is in the form of novel ophthalmic composition, preferably in the form of gel.

18: A novel pharmaceutical composition according to claim 6, wherein the pharmaceutical composition is in the form of novel ophthalmic composition, preferably in the form of gel.

19: A novel pharmaceutical composition according to claim 7, wherein the pharmaceutical composition is in the form of novel ophthalmic composition, preferably in the form of gel.

20: A novel pharmaceutical composition according to claim 8, wherein the pharmaceutical composition is in the form of novel ophthalmic composition, preferably in the form of gel.
